(19) United States
(12) Patent Application Publication
(10) Pub. No.: US 2020/0094245 A1
(43) Pub. Date: Mar. 26, 2020

(54) SAMPLE PRE-TREATMENT DEVICES AND METHODS

(71) Applicant: Universal Biosensors Pty Ltd, Rowville, Victoria (AU)

(72) Inventors: Garry Chambers, Melbourne (AU); Alastair M. Hodges, Blackburn South (AU)

(21) Appl. No.: 16/497,276

(22) PCT Filed: Mar. 24, 2018
(86) PCT No.: PCT/AU18/50273

§ 371 Date: Sep. 24, 2019

Related U.S. Application Data
(60) Provisional application No. 62/476,603, filed on Mar. 24, 2017.

(51) Int. Cl. B01L 3/00; C12Q 1/6806

(52) U.S. Cl. CPC B01L 3/502; C12Q 1/6806; B01L 2300/1805; B01L 2200/10; B01L 2200/16

(57) ABSTRACT

The invention disclosed herein relates to a sample treatment device. The sample treatment device can include a bore comprising at least one chamber, wherein the at least one chamber, or multiple chambers, is formed by using partitions to define the chamber inside the device bore. The chambers can be formed by having a single shaft that is adapted to be moved longitudinally through the bore of the device, the shaft having sealing elements formed on or as part of the shaft, wherein the sealing elements create a seal with the interior bore wall, wherein the separate chambers defined by the interior bore wall and the sealing elements are formed by the spaces between the sealing elements. The chambers of the sample treatment device disclosed herein can be suitable for performing mixing, chemical reaction, heating, cooling, separation and/or washing steps using a simple to manufacture device, where one or all of the steps can be conducted for a desired or predetermined time.

### CROSS REFERENCE TO RELATED APPLICATIONS

This application claims the benefit of U.S. Provisional Application No. 62/476,603, filed Mar. 24, 2017, entitled SAMPLE PRE-TREATMENT DEVICES AND METHODS. The entire contents of the foregoing are hereby incorporated by reference herein.

### BACKGROUND TO THE INVENTION

When testing a sample to measure a property of it, such as the presence or concentration of a substance in the sample, it is often necessary to treat the sample to render it suitable for use with the property measuring method. Samples are often treated to remove interfering species and/or to perform one or more conversion steps of a component of the sample. For example, when measuring the concentration of substances in blood, the sample is often treated to remove cells to produce plasma or serum prepared. This is often to remove potential species that interfere with the analysis methodology, such as red cells interfering with optical measurements. In tests looking for particular sequences of DNA or RNA in a sample it is often necessary to lyse the cells to liberate the DNA and then perform additional steps to separate or capture the DNA of interest. Likewise, in tests looking for a particular protein in a sample, it is often necessary to lyse the cells to expose the protein to reagents and/or to remove interfering species.

Sample treatment can be a particular challenge when required as part of a point of care assay due to the limited complexity of equipment available and the simplicity of steps the user is required to perform to make the test suitable for unskilled or semi-skilled users.

The instant application discloses a simple sample treatment device that can be used alone, or optionally integrated with an analysis portion or device that receives the output of the sample treatment device. The device is designed to allow for wet and/or dry reagents required to treat the sample, and optionally wet and/or dry reagents required to perform a sample analysis. The device can optionally allow mixing, heating or cooling of the sample and/or allow other steps required to perform a desired sample treatment. The device disclosed herein can be designed for a single use, can be inexpensive to fabricate, can require minimal user steps and a low complexity of auxiliary equipment, and can be easily integrated to a suitable analysis part. Methods using the device are also disclosed herein.

### SUMMARY OF INVENTION

The sample treatment device described herein includes a bore comprising at least one chamber, wherein the at least one chamber, or multiple chambers, is formed by using partitions to define the chamber inside the device bore. The partitions separating the at least one chamber from the rest of the bore of the device are sealing elements that form a seal with the bore wall, thus sealing one chamber from another and from the remainder of the bore. One or more openings in the wall of the bore of the device can be incorporated to allow ingress and egress of liquids and gases from the bore. The chambers of the device disclosed herein can be adapted to carry out various sample treatment steps, for example, but not limited to, mixing, chemical reaction, heating, cooling, separation, washing steps, and the like, or combinations thereof, in assays that require at least one sample treatment step as part of the analysis. For example, the sample treatment device disclosed herein can be used for performing sample treatment steps, such as, to remove potential species that interfere with analysis methodology from a sample and/or to perform one or more conversion steps of a component of a sample. For example, the chambers of the sample treatment device disclosed herein can be adapted to treat blood samples to remove red cells, to produce plasma, or to prepare serum, and the like. In some embodiments, a chamber of the device can be used to lyse cells in a sample to liberate the DNA, to perform steps to separate or capture DNA of interest, and/or to lyse the cells in a sample to expose protein to reagents.

In one embodiment, the bore of the device is circular in cross-section, such as a barrel, and the sealing elements are circular plates with a material at their circumference that is pliable enough to create at least a seal to a liquid when it pushes against sealing elements or the bore wall. For example, the bore can be a barrel. The plates can be made from a single material or from a combination of materials. In one embodiment, the body plate can comprise a stiff material with a different pliable material at its circumference. In another embodiment the plates can be made from a single material that is suitable to form the body and circumference of the plate. Examples of suitable material for the sealing elements include, for example, but are not limited to, polymers such as polyethylene, polypropylene, polyurethane, fluorinated polymers, polyester, nylon, viton, silicone rubbers, latex rubber and butyl rubbers, and the like, and/or combinations thereof. Additional examples of suitable material for the sealing elements include, for example, but are not limited to, metals such as stainless steel, copper, steel, brass, tin, nickel, or ceramics, or the like, and/or combinations thereof.

In one embodiment, the bore of the device includes a first chamber into which the sample to be treated can be introduced through an opening in the bore wall. In some embodiments, the bore of the device further includes a second chamber. In some embodiments, the device includes multiple chambers. In some embodiments, the device includes 1, 2, 3, 4, 5, 6 or more chambers. The chambers are formed by having a single shaft that is adapted to be moved longitudinally through the bore of the device, the shaft having sealing elements formed on or as part of the shaft, wherein the sealing elements create a seal with the interior bore wall, wherein the separate chambers defined by the interior bore wall and the sealing elements are formed by the spaces between the sealing elements. In some embodiments, the chamber volumes can range from about 10 microliter to about 1000 microliter.

In one embodiment, one or more chambers contain dried or liquid reagents suitable for carrying out the desired sample treatment. Optionally, one or more chambers further include a mixing means to facilitate mixing of the reagents with the sample. In some embodiments, one or more chambers include an assay liquid or other suitable medium for carrying out the desired analysis of the sample.

In one embodiment of use of the device, the shaft is positioned in relation to the body of the device such that a first chamber is aligned with a port through the bore wall through which the sample to be treated can be introduced into the first chamber. Once introduced, the sample reacts with reagents in the first chamber to begin sample treatment. The shaft is then moved longitudinally along the bore to a predetermined second position, whereupon sample treatment is continued. Optionally, the shaft can be moved to further predetermined positions of multiple chambers for further treatment steps.

In some embodiments, once all the sample treatment steps have been completed, the shaft is moved to a predetermined output position wherein the chamber is aligned with the output port, and whereupon the treated sample, or a component of the treated sample, is transported through the output port to the analysis part for analysis. The sample can be transferred using, for example, but not limited to, gravity, capillary force, pressure applied to a separate opening to the pre-treatment chamber, or other means, or a combination of such means.

In one embodiment, the sample treatment device is designed to be integrated to an analysis portion or device, where the output of the sample treatment device is transferred to the analysis portion or device for the desired analysis. The integration step can be performed by the user or the sample treatment device described herein and the analysis portion can be integrated during manufacture and supplied to the user as a single item.

In some embodiments, the device includes multiple chambers. Optionally, the final chamber of a multi-chamber device contains an assay liquid, such that when the shaft is moved to a predetermined position, the final chamber is aligned with an output port. In this position, if an analysis part is integrated, the assay liquid travels to fill the desired portions of the analysis part.

In some embodiments, the shaft can be positioned in the bore of the sample treatment device such that the first chamber is sealed from the rest of the bore and from any ports in the bore. This positioning can be set during manufacture of the device and maintained up until the point the device is used. This embodiment can be particularly advantageous if liquid reagents are to be incorporated into the sample pre-treatment chamber during device manufacture and maintained as liquids during device storage prior to use. It can also be advantageous when using dry reagents if, for example, it is desirable to maintain a desired level of dryness of the reagents prior to use.

The invention described herein relates to a sample treatment device. In some embodiments, the device includes a bore having proximal and distal ends, a longitudinal channel there through, having an opening for receiving a shaft at the proximal end, a sample port along the length of the bore at the proximal end for receiving a sample, at least one vent along the length of the bore, and an output port along the length of the bore at the distal end for releasing a treated sample. The device further includes a shaft including at least a first and second sealing element in fixed positions, wherein the sealing elements are adapted to conform to an inner surface of the bore thereby forming a seal in the longitudinal channel. When the shaft is inserted into the proximal end of the bore a first chamber is defined by the first and second sealing element and the inner surface of the bore is formed. The first chamber includes at least one means to treat a sample. The shaft is adapted to move along said longitudinal channel toward the distal end. In some embodiments, the shaft further includes a mixing element positioned between said first and second sealing element. The mixing element can be designed to agitate the liquid as the shaft is rotated relative to the device body. In some embodiments, the shaft is capable of being rotated to provide mixing of the sample by the mixing feature. In some embodiments, the sample treatment device can be interfaced to a simple auxiliary device to automate the steps carried out by the device

In some embodiments, the device can further include an assay device or a means at the distal end for attaching an assay device to the output port.

In some embodiments, the shaft can further include a reagent element positioned between a first and a second sealing element, wherein the reagent element includes at least one means to treat a sample. In some embodiments, the means to treat a sample includes a reagent. In some embodiments, the reagent to treat a sample includes a lysing agent. In some embodiments, the means to treat a sample includes magnetic beads coated with covalently attached oligonucleotides.

In some embodiments, one or more chambers are capable of being exposed to a heating means or a cooling means in a predetermined position. In some embodiments, the one or more chambers are capable of being exposed to a sonication means in a predetermined position.

In some embodiments, the shaft can further include a third sealing element, where a second chamber is defined by the second and third sealing element and the inner surface of the bore is formed. In some embodiments, the second chamber includes an assay liquid. In some embodiments, the assay liquid is transferred to the output port when the shaft is positioned such that the second chamber aligns with the output port.

In some embodiments, the shaft is adapted to move along said longitudinal channel from a storage position, to a loading position, to an output position. In some embodiments, in a storage position, the shaft is positioned such that the first chamber is not aligned with the vent, sample port or output port. In some embodiments, in the loading position, the shaft is positioned such that the first chamber is aligned with the sample port. In some embodiments, in the output position, the shaft is positioned such that the first chamber aligns with the output port.

In some embodiment, the device can further include an auxiliary device.

In some embodiments, the sample treatment device can include one, two, three, four, five or more chambers to carry out multiple sample treatment steps. For example, in the case of a DNA analysis, a first chamber of the sample treatment device disclosed herein can be adapted to receive a sample and liberate double stranded DNA from the sample, a second chamber can be adapted to convert the double stranded DNA to single stranded DNA, a third chamber can be adapted to collect and concentrate the single stranded DNA, a fourth chamber can be adapted to wash the single stranded DNA, and the washed and concentrated single stranded DNA can then be transported from the sample treatment device to an analysis part.

Some embodiments of the invention relate to methods for treating a sample using the sample treatment device. For example, one embodiment of the invention relates to a method for treating a sample, wherein the method includes adding a sample to the sample port of the device, and moving the shaft along the longitudinal channel toward the distal end thereby moving the sample through the one or more chambers of the sample treatment device, wherein one or more sample treatment steps are carried out in the one or more chambers of the sample treatment device.

In one embodiment of the method, one or more chambers of the sample treatment device contain dried or liquid reagents suitable for carrying out the desired sample treatment. In some embodiments, one or more chambers include an assay liquid or other suitable medium for carrying out the desired analysis of the sample. In some embodiments, the method further includes rotating the shaft in one or more chambers to facilitate mixing of the reagents with the sample.

In some embodiments, the method further includes heating or cooling one or more chambers at a predetermined position. In some embodiments, the method further includes exposing one or more chambers to a sonication means in a predetermined position.

In some embodiments, the method further includes moving the shaft to a predetermined output position, wherein the chamber is aligned with the output port, and transporting the treated sample, or a component of the treated sample through the output port to an analysis part for analysis. The sample can be transferred using, for example, but not limited to, gravity, capillary force, pressure applied to a separate opening to the pre-treatment chamber, or other means, or a combination of such means.

### BRIEF DESCRIPTION OF THE DRAWINGS

FIG. 1 is schematic of an embodiment of the invention showing three possible positions of the device.

FIG. 2 shows a top view of the device depicting the ports 7, 8, 9 and 10.

FIG. 3 is schematic of an embodiment of the invention showing four possible positions of the device.

FIG. 4 is schematic of an embodiment of the invention showing the device in a storage position.

### DETAILS OF INVENTION

It will be appreciated by one skilled in the art that the disclosed sample treatment device alone and in conjunction with and analysis part and auxiliary device will have multiple uses in assays that require at least one sample treatment step as part of the analysis. Note that in this disclosure, use of the term pre-treatment is not intended to imply only steps that strictly need to be carried out before an assay can be performed. It is intended that the definition of pre-treatment also includes steps that can be an integral part of assay procedure.

The invention disclosed herein is suitable for performing mixing, chemical reaction, heating, cooling, separation and/or washing steps using a simple to manufacture device, where one or all of the steps can be conducted for a desired or pre-determined time. The device can be conveniently manufactured utilizing technology commonly used in the manufacture of disposable plastic syringes. It can be configured to transport the desired pre-treated material to an integrated or separate analysis part. It can be interfaced to a simple auxiliary device to automate the steps carried out by the device, such that a user of the device is required to have minimal or no interaction with the device as it is performing the required steps.

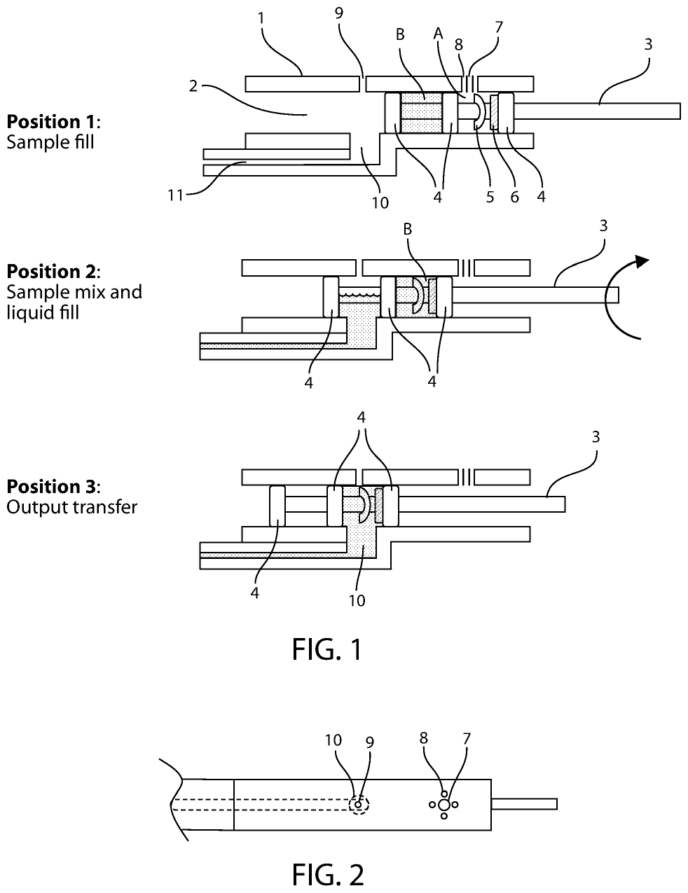

FIGS. 1 and 2 show schematics of an embodiment of the invention and can be used to illustrate device features.

In FIG. 1A a device body 1 includes a bore 2 through which a shaft 3 is inserted. Two or more sealing elements 4 are spaced along the shaft 3 to form chambers A and B. Chamber A is the chamber into which the sample is to be introduced. In the embodiment shown in FIG. 1, chamber A includes an optional mixing element 5 and a reagent element 6. In the embodiment shown in FIG. 1, chamber B is loaded with assay liquid during the assembly of the device. Port 7 through the device body 1 creates an opening from the outside of the device body to the interior of the bore of the device body.

The chambers formed in the bore of the device can have a range of volumes, depending upon the assay to be performed, the sample to be used and the analysis part to which the pre-treatment device is interfaced. Suitable chamber volumes can typically range from about 10 microliter to about 1000 microliter. This volume range is not determined by limitations in the possible size the device can be made, but rather by what is convenient in applications for which the device can be used and can be determined by a person of ordinary skill in the art depending on the desired application. Examples of application specific requirements that would be a factor in choosing convenient dimensions are the volume of sample that it is desired to use, the volume of liquid needed to transfer the desired sample component to and operate any associated analysis part, and the desired overall length, breadth and width of the pre-treatment device and any auxiliary device to which it can interface.

The chamber volumes can be determined by choosing the cross-sectional area of the bore of the device in combination with the distance between the sealing elements and the space occupied by the shaft of the device and any associated mixing or other apparatus present in the chamber. For example, when the bore and shaft of the device are circular in cross-section and there are no additional element taking up volume in the chamber, to achieve a 10 microliter chamber volume with, for example a 1.5 mm diameter shaft and a 2.5 mm diameter bore, the distance between the sealing sections would be about 3.2 mm. In another example, when the bore and shaft of the device are circular in cross-section and there are no additional elements taking up volume in the chamber, to achieve a 10000 microliter chamber volume with, for example a 10 mm diameter shaft and a 20 mm diameter bore, the distance between the sealing sections would be 4.2 mm. In another alternative to achieve a 1000 microliter chamber volume the shaft could be 5 mm in diameter, the bore 12 mm in diameter with a distance between the sealing plates of 10.7 mm. One skilled in the art will appreciate that there are a wide range of dimensions that would be suitable for construction of the device, that the foregoing are merely examples for illustrative purposes. The high level of flexibility in how the device is designed and constructed provides utility in a wide range of applications.

The reagents in chamber A can be in liquid form or in solid form. Solid reagents can be incorporated into chamber A, for example, by injecting a liquid into the chamber then removing a solvent such that solid reagents remain, by directly injecting solid reagents into the chamber, by incorporating a separate reagent element on the shaft, or by other suitable means. Suitable separate reagent elements include, for example, but are not limited to, self-supporting dry reagents formed into an element, dry reagents mixed with a binder to form a self-supporting element, or dry reagents coated onto a solid non-porous or porous substrate, and the like. Non-limiting examples of non-porous substrate materials include, for example, but are not limited to, sheets of polyester, polycarbonate, polyurethane, silicone, or glass, and the like. Non-limiting examples of porous substrate materials, include, for example, but are not limited to, cellulose paper, microporous membranes or foamed materials.

The mixing element 5 can be in the form of, for example, a paddle or paddles, a spiral or rod, or other suitable form for mixing the sample and optional reagents. Materials suitable for the mixing element include, for example, but are not limited to, polyethylene, polypropylene, fluorinated polymers, polyester, polycarbonate, polyurethane, silicone, or glass, and the like, and combinations thereof. In one embodiment, the mixing element can include holes, pits, channels or other features that can receive and hold a volume of liquid. The reagent in liquid form can be brought into contact with these features by, for example, dipping, spraying or dropping such that after contact, the features retain reagent liquid. A suitable drying means can then be used to remove liquid to leave dry reagent coating the features. Suitable drying means include, for example, but not limited to, passive air drying, active drying using gas flow or heated gas, exposure to IR radiation, and other methods common to one skilled in the art. In this embodiment, the mixing element can also serve as a depository for dry reagent. An advantage of this embodiment is that movement of the mixing element to mix the sample can simultaneously serve to aid the dissolution and dispersion of the reagents.

In use, sample is introduced through port 7 into chamber A. Port 8 in the device body serves as a vent to let air in chamber A escape as it is displaced by sample liquid. The sample treatment reagents 6 dissolve in the sample in chamber A and begin the sample treatment process. The mixing element 5 is designed to agitate the liquid as the shaft 3 is rotated relative to the device body 1. The shaft 3 can be optionally rotated while it is in a first position (position 1) by a rotating means. Suitable rotating means can include, for example, but not limited to, an electric motor that is engaged with the end of the shaft 3, for example a stepper motor where the rotation of the motor can be controlled in steps. Additional means can be means linking a user action to the rotation of the shaft 3, for example a wheel on the end of the shaft 3 that the user can rotate or a button that the user can push that causes the shaft to rotate through levers or gears.

After the desired time and initial sample treatment steps have been carried out, the shaft 3 is moved by a moving means to a second position (position 2) (FIG. 1B). Moving means can be manual or mechanical or both. Examples of suitable mechanical means include, for example, but are not limited to, an electric motor, where the rotation of the motor causes movement of the shaft 3. This means can include rack and pinion gears or helical gears to translate the circular motion of the motor to a linear motion of the shaft 3. In one embodiment, a single motor is used to both rotate the shaft and move the shaft longitudinally, for example through the use of helical gears. In the second position (position 2), further sample treatment steps can optionally be carried out. In some embodiments, heat can be applied to the sample in chamber A when it is in the first position (position 1) as part of the sample treatment, wherein a heating means can apply heat to the sample, for example, through the walls of the device body 1, wherein the heating means is aligned with the first position (position 1). Examples of suitable heating means include, for example, but are not limited to, a heated metal or ceramic element brought close to or in contact with the body of the device, heating of air that is brought into contact with the device, IR radiation that is shone onto the device, and other similar means. In this embodiment, when the shaft is moved to the second position (position 2), the sample is moved out of the heated zone and can be cooled as a subsequent sample treatment step. The shaft can be optionally rotated in the second position (position 2). In other embodiments, the sample can be sonicated while in this position, for example by a sonication means brought into contact with the device body that transfers sonic energy through the device body to the sample.

As shown in FIG. 1B, in the second position (position 2), chamber B is brought into alignment with output port 10. In this position, assay liquid in chamber B, if included, is transferred to the assay part (not shown) through channel 11. Optional port 9 provides an opening to allow air to enter to replace the assay buffer transferred. In some embodiments, port 9 is not present as a small enough volume of liquid is transferred, or a sealing plate of chamber B is flexible enough, so as to not require air to replace the transferred liquid. The sonic wave can for example lyse cells in the sample, heat the sample or cause chemical reactions to take place in the sample.

In a third position (position 3) (FIG. 1C), chamber A is bought into alignment with output port 10. As shown in FIG. 1C, in the third position (position 3), a component of the material in chamber A being transferred to output channel 11. Optional port 9 provides an opening to allow air to enter to replace the contents transferred.

FIG. 2 shows a top view of one embodiment of the device depicting the ports 7, 8, 9 and 10.

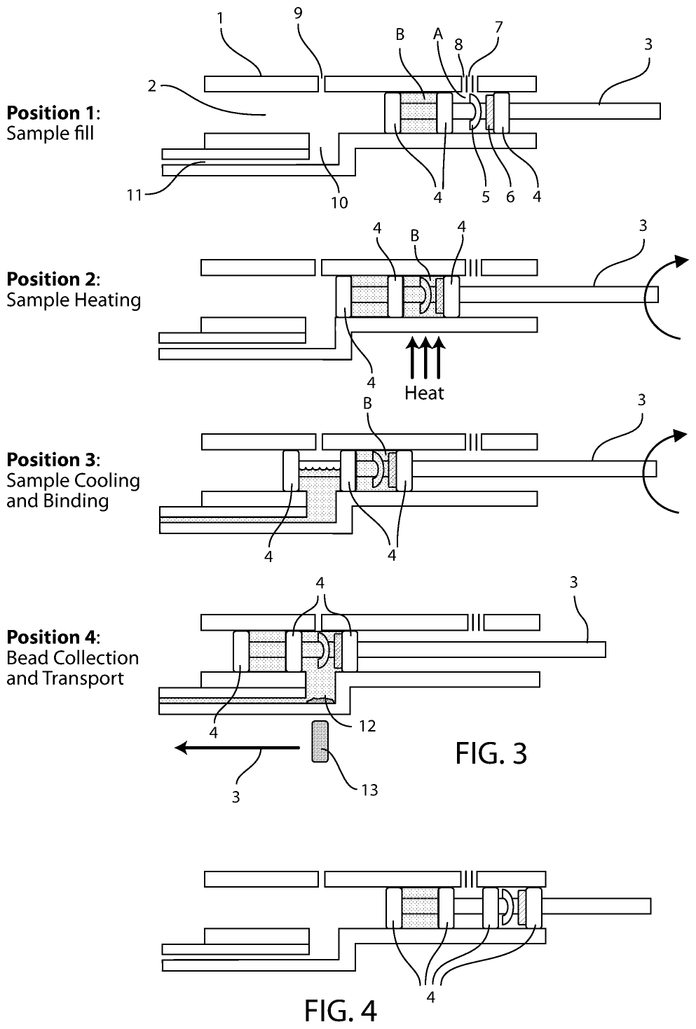

Optionally, there can be at least one additional position between the sample introduction position and the second position in FIG. 1. An embodiment with this additional position is shown in FIG. 3.

In one embodiment of the device, the sample pre-treatment chamber A is not in alignment with port 7 or port 8 prior to the device being used, but rather is positioned not to be aligned with any ports. For example, chamber A can be positioned in a section of the bore to the right of the first position (position 1) shown in FIGS. 1 and 3, such that the chamber A is closed off from ports 7 and 8. This can constitute a storage position for the shaft, that is, a position of the shaft that is maintained prior to use of the device. In this embodiment the shaft can be moved from the storage position to an initial use position as a step in the device use. In embodiments including a storage position that include more than one chamber containing liquids or dry reagents prior to use, the device can be configured such that in the storage position one or more or all of these additional chambers can be positioned so as not to align with any port in the device, thereby sealing the chambers off when in the storage position. FIG. 4 shows an example of this embodiment of the device with the shaft in the storage position.

In another embodiment of the device, a chamber between a dry reagent containing chamber and a liquid containing chamber can be formed. An example of such a chamber is shown in FIG. 4 as the intermediate space between the leftmost and the rightmost chambers. It can optionally be arranged such that when the shaft 3 is in a storage position, none of the reagent containing chamber, the liquid containing chamber and the intermediate chamber align with any ports in the bore of the device. In these embodiments, desiccant material can be incorporated into the intermediate chamber, where the purpose of the desiccant material can be to assist in maintaining the dryness of the reagents in the reagent chamber. The desiccant material is a material that can absorb moisture from the surrounding environment. Examples of suitable desiccant materials include, but are not limited to, molecular sieve, activated carbon, silica gel, and the like. The desiccant can be, for example, in the form or a powder, pellets, sheet or block. Optionally, the desiccant material can be mixed with a binder material to form a composite structure with improved handling properties. Examples of suitable binder materials include, but are not limited to, polymers, such as thermoplastic polymers, for example, polyethylene or polypropylene. In addition to or instead of the desiccant being incorporated into the sample treatment device, the sample treatment device can be stored in external packaging that is resistant to water vapor transmission, and optionally comprising desiccant material. Examples of suitable desiccant materials are those disclosed above for incorporation into the sample treatment device intermediate chamber. Suitable packaging material are those commonly known in the art such as for example, but not limited to, metal foil, plastic sheet coated with a metal layer, or other material that creates a suitable barrier to water vapour transmission. In one example embodiment, desiccant can be incorporated into an intermediate chamber in the sample treatment device, where the intermediate chamber is aligned with a port in the bore of the device when the shaft is in the storage position. The device can be packaged in external packaging that is resistant to water vapor transmission. The desiccant can then serve to maintain a reduce humidity in the sample treatment device and within the external packaging while the sample treatment device is stored.

The sample treatment device described herein can be interfaced to an auxiliary device by the user inserting it into the auxiliary device, whereupon the free end of the shaft of the device can be engaged with a motorized drive, where the motorized drive is designed to advance the shaft to pre-determined positions at pre-determined times and, optionally, rotate the shaft and/or provide mixing of the sample. If the pre-treatment device is integrated to an analysis part or the user introduces a separate analysis part into the auxiliary device, then the auxiliary device can also include the means to run the desired assay. For example the auxiliary device can include the means to conduct an optical assay in conjunction with the assay part or can include the means to conduct an electrochemical assay in conjunction with the assay part. Optionally the auxiliary device can be controlled by a microcontroller and include, heating means, cooling means, means for detecting the presence of sample in the pre-treatment device or other means necessary or desirable in conducting the desired sample pre-treatment and analysis steps. In addition, the auxiliary device can include means for analyzing the signal from the analysis part, displaying and storing the analysis result and interfacing with other equipment to which information generated by the auxiliary device can be transferred. By using a suitable auxiliary device in conjunction with the sample treatment device described herein, the user can introduce the sample into the device, with the other steps automated by the device, to be able to obtain the desired analysis result.

This device can be suitable for concentrating a component of the sample and transferring the component in concentrated form to an analysis part. In embodiments of the device according to this use, the sample pre-treatment can include attaching the desired component of the sample to a substrate, the substrate collected from the sample and transferred to the outlet port of the device. Suitable substrates have the properties that the component of interest can be attached to them and that means can be employed to collect them and transport them to an analysis part. Examples of suitable substrates are polymer beads comprising a surface to which the sample component of interest can attach and where, for example, gravity or centrifugal force can be used to collect the beads. In one embodiment, the substrate can be magnetic beads comprising a surface to which the sample component of interest can attach, where an external magnetic field can be applied to collect and transport the substrate. In other embodiments, the substrate can be in the form of bead or other solid that are denser than the sample, where the shaft can be rotated to generate centrifugal force to collect the bead or other solid.

Examples of suitable coatings for the substrate to allow for attachment of the component of interest are antibodies covalently attached to the surface, oligonucleotides, haptens, or other species that facilitate attachment of the component of interest. If, for example, the component of interest is a DNA or RNA molecule with a particular sequence of bases, the coating on the substrate can include a DNA molecule with a complementary sequence to the sequence of interest.

### EXAMPLES

The following non-limiting example is provided to further illustrate embodiments of the invention described herein. It should be appreciated by those of skill in the art that the techniques disclosed in the examples that follow represent approaches discovered by the inventors to function well in the practice of the application, and thus can be considered to constitute examples of modes for its practice. However, those of skill in the art should, in light of the instant disclosure, appreciate that many changes can be made in the specific embodiments that are disclosed and still obtain a like or similar result without departing from the spirit and scope of the application.

### Example 1

In one embodiment, DNA is the component of interest and a magnetic bead substrate is used to collect the DNA. The steps in this example are represented schematically in FIG. 3.

In this example, with the shaft in first position (position 1) (FIG. 3A), an aliquot of the whole blood, urine or other fluid to be treated is introduced into chamber A via port 7 with the displaced air venting through port 8. The reagents 6 in chamber A include a lysing agent and magnetic beads coated with covalently attached oligonucleotides. The lysing agent lyses cells in the sample to liberate any DNA present. The oligonucleotide coating the magnetic beads include a sequence that is complementary to a sequence in the DNA component of interest.

The shaft is then advanced to the second position (Position 2) shown in FIG. 3B whereupon it is optionally rotated to promote mixing of the reagent and lysing of the cells. Heat is applied in this position to convert the double stranded DNA in the sample to single stranded DNA.

The shaft is then advanced to the third position (position 3) shown in FIG. 3C where it can be optionally rotated to further promote mixing. In this position the sample is allowed to cool, promoting the now single stranded DNA of interest to hybridize to oligonucleotides on the magnetic beads. In this position, assay buffer from chamber B is transferred to fill output port 10 and channel 11. The assay buffer is transferred using gravity, capillary force, pressure applied to port 9, or other means or a combination of such means. Port 9 provides an opening to allow air to enter to replace the assay buffer transferred.

The shaft is then advanced to the fourth position (position 4) shown in FIG. 3D, whereupon the magnetic field from a magnet 13 draws the magnetic beads 12 to the base of the output port 10. The magnet 13 can then be moved in the direction of the arrow 14 to transport the magnetic beads 12 along channel 11 to the analysis part. The assay buffer filing output port 10 and channel 11 acts to wash the magnetic beads and associated DNA as they are transported into the port and along the channel, separating them from other components of the sample.

In this example, the device liberates double stranded DNA, converts it to single stranded DNA, collects and concentrates it, washes it and transports the washed and concentrated DNA to an analysis part.

### Example 2

In another example, the pre-treatment device is used where the sample is required to be pre-reacted with one or more reagents to be suitable for analysis, for example, where a pre-reaction is desired to remove interfering species from the sample or to perform a conversion step of a component of the sample. The reaction can be a reaction to convert a component to another chemical species or it can, for example, be an absorption reaction, where undesirable species are removed from the sample by being absorbed into or onto a solid reagent in the pre-treatment chamber. Embodiments suitable for use with these examples can be ones with only a single chamber in the device, which is the pre-treatment chamber.

In this embodiment, the pre-treatment chamber is advanced from its storage position to align with a sample port, such as port 7 in FIG. 1, whereupon the user introduces sample into the device. The shaft is then optionally advanced to a mixing and reaction position where the shaft can be rotated to mix the sample and other means, such as heating, applied as desired to affect the required sample pre-treatment. The shaft is then advanced to another position where the pretreatment chamber is brought into alignment with an output port, for example shown as 10 in FIG. 1. In this position the treated sample is transferred via the output port to the sample analysis part. The sample can be transferred using, for example, gravity, capillary force, pressure applied to a separate opening to the pre-treatment chamber, such as port 9, or other means or a combination of such means.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the invention disclosed herein are illustrative of the principles of the embodiments of the invention. Other modifications that can be employed can be within the scope of the invention. Thus, by way of example, but not of limitation, alternative configurations of the embodiments of the invention can be utilized in accordance with the teachings herein. Accordingly, embodiments of the present invention are not limited to that precisely as shown and described.

1. A sample treatment device, comprising:
a bore having proximal and distal ends, a longitudinal channel therethrough, having an opening for receiving a shaft at the proximal end, a sample port along the length of the bore at the proximal end for receiving a sample, at least one vent along the length of the bore, and an output port along the length of the bore at the distal end for releasing a treated sample;
a shaft comprising at least a first and second sealing element in fixed positions, wherein the sealing elements are adapted to conform to an inner surface of the bore thereby forming a seal in the longitudinal channel;
wherein when the shaft is inserted into the proximal end of the bore a first chamber is defined by the first and second sealing element and the inner surface of the bore is formed;
wherein the first chamber comprises at least one means to treat a sample; and
wherein the shaft is adapted to move along said longitudinal channel toward the distal end.

2. The device of claim 1, further comprising an assay device.

3. The device of claim 1, wherein the shaft further comprises a mixing element positioned between said first and second sealing element.

4. The device of claim 3, wherein the shaft is capable of being rotated to provide mixing of the sample by the mixing feature.

5. The device of claim 1, wherein the means to treat a sample comprises a reagent.

6. The device of claim 5, wherein the reagent to treat a sample comprises a lysing agent.

7. The device of claim 1, wherein the means to treat a sample comprises magnetic beads coated with covalently attached oligonucleotides.

8. The device of claim 1, wherein the means to treat a sample comprises a means for applying heat or sonication to the sample.

9. The device of claim 1, wherein the first chamber is capable of being exposed to a heating means in a predetermined position.

10. The device of claim 1, wherein the first chamber is capable of being exposed to a sonication means in a predetermined position.

11. The device of claim 1, the shaft further comprising a third sealing element, wherein a second chamber defined by the second and third sealing element and the inner surface of the bore is formed.

12. The device of claim 11, wherein the device further comprises a reagent element positioned the first or the second chamber, wherein the reagent element comprises at least one reagent to treat the sample.

13. The device of claim 11, wherein the second chamber comprises an assay liquid.

14. The device of claim 13, wherein the assay liquid is transferred to the output port when the shaft is positioned such that the second chamber aligns with the output port.

15. The device of claim 1, wherein the shaft is adapted to move along said longitudinal channel from a storage position, to a loading position, to an output position.

16. The device of claim 15, wherein in a storage position, the shaft is positioned such that the first chamber is not aligned with the vent, sample port or output port.

17. The device of claim 15, wherein in the loading position, the shaft is positioned such that the first chamber is aligned with the sample port.

18. The device of claim 15, wherein in the output position, the shaft is positioned such that the first chamber aligns with the output port.

19. The device of claim 1 further comprising an auxiliary device.

20. A method for treating a sample comprising adding a sample to the sample port of claim 1 and moving the shaft along the longitudinal channel toward the distal end thereby moving the sample through the one or more chambers of the sample treatment device, wherein one or more sample treatment steps are carried out in one or more chambers of the sample treatment device.
